(19) United States
(12) Patent Application Publication
(10) Pub. No.: US 2011/0245294 A1
(43) Pub. Date: Oct. 6, 2011

(54) METHODS OF IMPROVING QUALITY OF SLEEP

(75) Inventors: Mehdi PABORJI, San Mateo, CA (US); Roger S. FLUGEL, San Mateo, CA (US)

(73) Assignee: THERAVIDA, INC., San Mateo, CA (US)

(21) Appl. No.: 13/078,826

(22) Filed: Apr. 1, 2011

Related U.S. Application Data
(60) Provisional application No. 61/320,208, filed on Apr. 1, 2010.

(51) Int. Cl. A61K 31/439; A61K 31/4178; A61K 31/24; A61P 13/10

(52) U.S. Cl. 514/305; 514/397; 514/534

(57) ABSTRACT

Disclosed herein are methods of treating a patient suffering from overactive bladder (OAB) comprising administering to the patient a combination of antimuscarinic or anticholinergic agent and muscarinic agonist for the treatment of poor quality of sleep in the OAB patient.

### RELATED APPLICATIONS

This application claims priority to U.S. Provisional Application No. 61/320,208, filed Apr. 1, 2010, by Mehdi Paborji, and entitled “METHODS OF IMPROVING QUALITY OF SLEEP,” which is incorporated herein by reference in its entirety.

### FIELD OF THE INVENTION

The present invention is in the field of methods of using pharmaceutical compositions for improving the quality of sleep in patients on antimuscarinic therapy.

### BACKGROUND OF THE DISCLOSURE

Patients suffering from overactive bladder (OAB) also complain about poor quality of sleep. This may be attributed to nocturia, or nighttime urinary frequency that disturbs sleep (more than twice a night). There may also be some biochemical basis for the lack of quality of sleep in these patients. For example, some studies have suggested that oxybutynin, tolterodine, and trospium can have adverse effect on the sleep of healthy volunteers.

Several classes of medications have been used to treat and manage OAB. A recent evidence-based systematic review of controlled clinical trials of several agents concluded that these therapies significantly improved several indices of lower urinary tract function, including frequency of micturitions, nocturia and number of incontinence episodes. A major limitation of these agents is that they do not remedy the symptom of poor quality of sleep.

Thus, there exists a need in the art for a medication that provides sufficient efficacy for the treatment of OAB symptoms including poor quality of sleep in order to increase patient compliance, comfort, and efficacy.

### SUMMARY OF THE INVENTION

Disclosed herein are methods of improving quality of sleep in a patient suffering from overactive bladder, the method comprising: (a) identifying a patient in need thereof, and (b) administering to the patient a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof, wherein the first compound is an antimuscarinic or an anticholinergic agent and the second compound is a muscarinic agonist, and whereby the quality of sleep in the patient is improved.

Also disclosed are methods of improving quality of sleep in a patient suffering from nocturia, the method comprising: (a) identifying a patient in need thereof; and (b) administering to the patient a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof, wherein the first compound is an antimuscarinic or an anticholinergic agent, the second compound is a muscarinic agonist, and whereby the quality of sleep in the patient is improved.

Further disclosed herein are methods of improving quality of sleep in a patient being treated for overactive bladder by administration of a first compound, the method comprising: (a) identifying a patient in need thereof, and (b) administering to the patient a therapeutically effective amount of a second compound, while continuing the administration of therapeutically effective amount of the first compound, wherein the first compound is an antimuscarinic agent or an anticholinergic agent, wherein the second compound is a muscarinic agonist, and whereby the quality of sleep in the patient is improved.

In addition, disclosed herein are methods of improving quality of sleep in a patient suffering from nocturia, and being treated for overactive bladder by administration of a first compound, the method comprising: (a) identifying a patient in need thereof, and (b) administering to the patient a combination of a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof, wherein the first compound is an antimuscarinic or an anticholinergic agent and the second compound is a muscarinic agonist, whereby the quality of sleep in the patient is improved.

### DETAILED DESCRIPTION OF THE EMBODIMENTS

One of the major limitations of the administration of muscarinic or cholinergic antagonists is the resulting poor quality of sleep. Patients suffering from overactive bladder (OAB) suffer from this side effect more than others on antimuscarinic or anticholinergic treatments because the sleep pattern of OAB patients is further broken due to repeated night time bladder relieving. Thus, the quality of life of all patients on antimuscarinic or anticholinergic treatments, and in particular OAB patients, is hampered significantly to the extent that majority of patients discontinue the medications after about 2-6 months.

Thus, in the first aspect, disclosed herein are methods of treating a patient comprising administering to a patient in need thereof a therapeutically effective amount of a first compound and a therapeutically effective amount of a second compound, wherein the first compound is an antimuscarinic or an anticholinergic agent and the second compound is a muscarinic agonist and whereby the quality of sleep is improved.

Within the context of the present disclosure, a “muscarinic agonist” is a compound that modulates, i.e., agonizes, the activity of a muscarinic receptor either directly or indirectly. A muscarinic agonist acts directly on the muscarinic receptors when the muscarinic agonist itself binds to the muscarinic receptor and modulates its activity. A muscarinic agonist acts indirectly on the muscarinic receptors when the muscarinic agonist stimulates the production of an endogenous muscarinic agonist, which in turn agonizes the muscarinic receptors. An endogenous muscarinic agonist is a natural binding partner of the muscarinic receptors and is produced by the body of the subject itself. An example of an endogenous muscarinic agonist is acetylcholine.

In another aspect, disclosed herein are methods of improving quality of sleep in a patient suffering from nocturia, the method comprising: (a) identifying a patient in need thereof; and (b) administering to the patient a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof, where the first compound is an antimuscarinic or an anticholinergic agent, the second compound is a muscarinic agonist, and whereby the quality of sleep in the patient is improved.

In yet another aspect, disclosed herein are methods of improving quality of sleep in a patient being treated for overactive bladder by administration of a first compound, the method comprising: (a) identifying a patient in need thereof, and (b) administering to the patient a therapeutically effective amount of a second compound, while continuing the administration of therapeutically effective amount of the first compound, where the first compound is an antimuscarinic agent or an anticholinergic agent, where the second compound is a muscarinic agonist, and whereby the quality of sleep in the patient is improved.

In still another aspect, disclosed herein are methods of improving quality of sleep in a patient suffering from nocturia, and being treated for overactive bladder by administration of a first compound, the method comprising: (a) identifying a patient in need thereof, and (b) administering to the patient a combination of a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof, where the first compound is an antimuscarinic or an anticholinergic agent and the second compound is a muscarinic agonist, whereby the quality of sleep in the patient is improved.

The first compound of the methods described herein is a compound useful in the treatment of overactive bladder. In some embodiments, the first compound is an antagonist on one or more subtypes of muscarinic receptors. In further embodiments, the first compound may be selected from the group consisting of oxybutynin, tolterodine, solifenacin, darifenacin, trospium, fesoterodine, propiverine, imidafenacin, and dicyclomine, a metabolite thereof, or a pharmaceutically acceptable salt or prodrug thereof. In some embodiments, oxybutynin is S-oxybutynin, while in other embodiments, oxybutynin is R-oxybutynin, and in yet other embodiments, oxybutynin is a mixture of the S and the R isomers, for example a racemic mixture. In some embodiments, the metabolite of oxybutynin is N-desethyloxybutynin. In some embodiments, the metabolite of tolterodine is an N-dealkylated tolterodine. In other embodiments, the metabolite of tolterodine is 5-hydroxymethyl tolterodine. Other compounds known now or later developed for the treatment of OAB are within the scope of the present disclosure.

In some embodiments, the first compound is a compound of Formula I

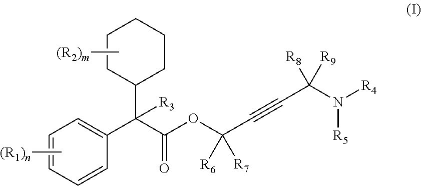

or a pharmaceutically acceptable salt or prodrug thereof, wherein:

R₁-R₉ are each independently selected from the group consisting of hydrogen, alkyl, nitro, amino, cyano, hydroxy, alkoxy, carboxylate, and amide; and

m and n are each independently selected from 1, 2, 3, 4, and 5.

In some embodiments, each R₁ and R₂ is independently selected from the group consisting of hydrogen, alkyl, hydroxy, and alkoxy. In certain embodiments, each R₁ and R₂ is hydrogen.

In some embodiments, R₃ is selected from the group consisting of hydrogen, alkyl, hydroxy, and alkoxy. In certain embodiments, R₃ is hydroxy.

In some embodiments, R₄ and R₅ are each independently selected from the group consisting of hydrogen, alkyl, hydroxy, and alkoxy. In certain embodiments, R₄ and R₅ are each independently an alkyl. In further embodiments, R₄ and R₅ are each independently selected from the group consisting of methyl, ethyl, propyl, n-butyl, isobutyl, and tert-butyl. In other embodiments, R₄ and R₅ are each independently ethyl.

In some embodiments, R₆-R₉ are each independently selected from the group consisting of hydrogen, alkyl, hydroxy, and alkoxy. In certain embodiments, R₆-R₉ are each independently a hydrogen.

In some embodiments, the first compound is oxybutynin, a free base thereof or a pharmaceutically acceptable salt or prodrug thereof. Oxybutynin is the active ingredient found in drugs such as Ditropan®, Ditropan XL®, Gelnique®, and Oxytrol®. In some embodiments, oxybutynin is present as the free base or as oxybutynin hydrochloride. Oxybutynin is an anticholinergic drug, thereby suppressing involuntary contractions of the bladder's smooth muscle. Oxybutynin is also believed to have muscarinic receptor activity, which further enhances its OAB efficacy. However, the same characteristics that render oxybutynin a successful therapeutic candidate for overactive bladder cause poor quality of sleep in the patients.

In some embodiments, the first compound is tolterodine, a free base thereof or a pharmaceutically acceptable salt or prodrug thereof. Tolterodine, which has the chemical name (R)-2-[3-[bis(1-methylethyl-amino]-1-phenylpropyl]-4-methylphenol [R—(R*,R*)]-2,3-dihydroxybutanedionic acid, is a muscarinic receptor antagonist and is the active ingredient found in drugs such as Detrol® (as tolterodine tartrate) and Detrol LA®. In another embodiment, the first compound is the 5-hydroxymethyl derivative of tolterodine.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, succinic acid, tartaric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

Throughout the present disclosure, when a particular compound is named, it is understood that the name refers to both the free base, or free acid, of the compound, and the pharmaceutically acceptable salts thereof. Thus, for example, the scope of the term “tolterodine” covers both the free base of tolterodine, i.e., (R)-2-[3-[bis(1-methylethyl-amino]-1-phenylpropyl]-4-methylphenol [R—(R*,R*)]-2,3-dihydroxybutanedionic acid, and its various pharmaceutically acceptable salts, for example tolterodine tartrate.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

In some embodiments, the second compound is a muscarinic agonist. In certain embodiments, the second compound is selected from the group consisting of pilocarpine, cevimeline, anethole trithione, aclatonium napadisilate, and yohimbine, or a pharmaceutically acceptable salt or prodrug thereof. In further embodiments, the second compound is pilocarpine, or a pharmaceutically acceptable salt or prodrug thereof. In other embodiments, the second compound is cevimeline, or a pharmaceutically acceptable salt or prodrug thereof.

In some embodiments, the second compound is a compound of Formula II

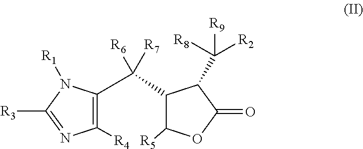

or a pharmaceutically acceptable salt or prodrug thereof, wherein

R₁-R₉ are each independently selected from the group consisting of hydrogen, alkyl, nitro, amino, cyano, hydroxy, alkoxy, carboxylate, and amide.

In some embodiments, R₁ and R₂ are each independently selected from the group consisting of hydrogen, alkyl, hydroxy, and alkoxy. In certain embodiments, R₁ and R₂ are each independently an alkyl. In further embodiments, R₁ and R₂ are each independently selected from the group consisting of methyl, ethyl, propyl, n-butyl, isobutyl, and tert-butyl. In other embodiments, R₁ and R₂ are each independently methyl.

In some embodiments, R₃-R₉ are each independently selected from the group consisting of hydrogen, alkyl, hydroxy, and alkoxy. In certain embodiments, R₃-R₉ are each independently a hydrogen.

Throughout the present disclosure, when a particular compound is mentioned by name, for example, oxybutynin, tolterodine, pilocarpine or cevimeline, it is understood that the scope of the present disclosure encompasses pharmaceutically acceptable salts, esters, amides, or prodrugs of the named compound. Also, if the named compound comprises a chiral center, the scope of the present disclosure also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the named compound comprises more than one chiral center, the scope of the present disclosure also includes compositions comprising a mixture of the various diastereomers, as well as compositions comprising each diastereomer substantially free of the other diastereomers. Thus, for example, commercially available oxybutynin is a racemic mixture comprising two separate enantiomers. The recitation of “oxybutynin” throughout this disclosure includes compositions that comprise the racemic mixture of oxybutynin, the compositions that comprise the (+) enantiomer substantially free of the (−) enantiomer, and the compositions that comprise the (−) enantiomer substantially free of the (+) enantiomer. Further, for example, commercially available pilocarpine, which is a naturally occurring alkaloid, comprises two stereocenters. The scope of the present invention includes pharmaceutical compositions comprising all four diastereomers, pharmaceutical compositions comprising the racemic mixture of R,R and S,S isomers, pharmaceutical compositions comprising the racemic mixture of R,S and S,R isomers, pharmaceutical compositions comprising the R,R enantiomer substantially free of the other diastereomers, pharmaceutical compositions comprising the S,S enantiomer substantially free of the other diastereomers, pharmaceutical compositions comprising the R,S enantiomer substantially free of the other diastereomers, and pharmaceutical compositions comprising the S,R enantiomer substantially free of the other diastereomers.

In certain embodiments, the present invention relates to a method of treating a patient comprising administering to a patient in need thereof a therapeutically effective amount of a combination selected from the group consisting of: oxybutynin and pilocarpine, oxybutynin and cevimeline, oxybutynin and anethole trithione, oxybutynin and aclatonium napadisilate, oxybutynin and yohimbine, tolterodine and pilocarpine, tolterodine and cevimeline, tolterodine and anethole trithione, tolterodine and aclatonium napadisilate, tolterodine and yohimbine, solifenacin and pilocarpine, solifenacin and cevimeline, solifenacin and anethole trithione, solifenacin and aclatonium napadisilate, solifenacin and yohimbine, darifenacin and pilocarpine, darifenacin and cevimeline, darifenacin and anethole trithione, darifenacin and aclatonium napadisilate, darifenacin and yohimbine, trospium and pilocarpine, trospium and cevimeline, trospium and anethole trithione, trospium and aclatonium napadisilate, trospium and yohimbine, fesoterodine and pilocarpine, fesoterodine and cevimeline, fesoterodine and anethole trithione, fesoterodine and aclatonium napadisilate, fesoterodine and yohimbine, propiverine and pilocarpine, propiverine and cevimeline, propiverine and anethole trithione, propiverine and aclatonium napadisilate, propiverine and yohimbine, imidafenacin and pilocarpine, imidafenacin and cevimeline, imidafenacin and anethole trithione, imidafenacin and aclatonium napadisilate, imidafenacin and yohimbine, dicyclomine and pilocarpine, dicyclomine and cevimeline, dicyclomine and anethole trithione, dicyclomine and aclatonium napadisilate, and dicyclomine and yohimbine.

The compounds useful for the methods described herein may be used in various formulations. Certain formulations affect the rate at which the compound enters the blood stream of the patient. Thus, some formulations are immediate release formulations while other formulations are delayed release, sustained release, or extended release formulations.

Thus, in some embodiments, the first compound is in immediate release formulation, while in other embodiments the first compound is in delayed release formulation, and in yet other embodiments the first compound is in sustained release formulation, and in further embodiments the first compound is in extended release formulation. In some embodiments, the second compound is in immediate release formulation, while in other embodiments the second compound is in delayed release formulation, and in yet other embodiments the second compound is in sustained release formulation, and in further embodiments the second compound is in extended release formulation. In some embodiments, the third compound is in immediate release formulation, while in other embodiments the third compound is in delayed release formulation, and in yet other embodiments the third compound is in sustained release formulation, and in further embodiments the third compound is in extended release formulation.

The methods described herein are particularly useful in alleviating the side effects in the treatment of OAB, namely poor quality of sleep, improving tolerability, and enhancing patient compliance while increasing the patient's quality of life.

A patient in need of the treatment methods disclosed herein may be a patient who suffers from overactive bladder. The patient may also be one who finds current therapies for overactive bladder uncomfortable and/or the unalleviated symptoms such as the poor quality of sleep, intolerable enough so as to require adjunct therapy. The patient may also be one who is considering discontinuing therapy for overactive bladder due to the unalleviated symptoms. In some embodiments, a patient who is recently diagnosed with overactive bladder but yet has not been treated therefore is a patient in need of the treatment methods and compositions disclosed herein. In these embodiments, the patient begins the therapy using the methods and combinations disclosed herein so that the patient does not experience any of the side effects, or experience the side effects to a lesser degree or the symptoms including poor quality of sleep is alleviated.

In some embodiments, the patient in need of the treatment methods disclosed herein may already be undergoing treated for OAB by administration of a therapeutically effective amount of antimuscuranic or anticholinergic agents. In other embodiments, the patient has not been treated for OAB.

In some embodiments, the patient may be suffering from overactive bladder, urge, stress, and mixed incontinence.

In some embodiments the first compound and the second compound are administered more or less simultaneously. In other embodiments the first compound is administered prior to the second compound. In yet other embodiments, the first compound is administered subsequent to the second compound.

It should be noted that simply taking commercially available pilocarpine HCl, e.g., Salagen® tablets, or any other muscarinic agonists in conjunction with an OAB drug is not effective to alleviate the symptom of poor quality of sleep. Certain effective treatments match the pharmacokinetic profile of each salivary gland stimulant, such as pilocarpine, cevimeline, anethole trithione, aclatonium napadisilate, or yohimbine, with the pharmacokinetic profiles of the OAB agents, for example oxybutynin, tolterodine, solifenacin, darifenacin, trospium, fesoterodine, propiverine, imidafenacin, and dicyclomine, and other approved agents or in development.

Therefore, in certain embodiments in the above methods, the first and second compounds are administered such that the peak plasma concentration for the first compound occurs at nearly the same time after administration as the peak plasma concentration for the second compound. Thus, the two compounds may be administered simultaneously, but be formulated such that the delay in their release causes the two peak plasma concentrations to occur simultaneously or at nearly the same time. In other embodiments, one compound is administered at a time interval after the other compound in order to ensure that the peak plasma concentrations occur at nearly the same time.

In other embodiments in the above methods, the first and second compounds are administered such that the time point at which the lowest saliva flow occurs because of the action of the first compound nearly corresponds to the time point at which the highest saliva flow occurs because of the action of the second compound. Thus, the two compounds may be administered simultaneously, but be formulated such that the delay in their release causes the peak saliva flow time point for the second compound to occur at nearly the same time as the lowest saliva flow time point for the first compound. In other embodiments, one compound is administered at a time interval after the other compound in order to ensure that peak and trough saliva flow time points match.

In some embodiments in the above methods, the first and second compounds are administered such that the ratio of their plasma concentrations, at a given point in time following their administration, is a predetermined value. Those of ordinary skill in the art recognize that the ratio of plasma concentrations is not necessarily the same as the ratio of the amount of compound administered. Compounds are dissolved differently in the gut, pass the gut wall differently, and have a different rate of first-pass metabolism in the liver. Furthermore, the clearance rate by the kidney is different for various compounds. Thus, for example, even if two compounds are administered in equivalent molar amounts, their plasma concentrations at a point in time after the administration may be significantly different. The methods disclosed herein take into account the pharmacokinetics of drug intake and metabolism, such that the ratio of the two compounds at the time of administration is adjusted so that the two compounds would have a predetermined concentration ratio in the plasma.

In some embodiments the dosage form is designed as sustained release of one agent combined with either sustained release or immediate release of the second agent to ensure that the peak plasma concentrations occur at nearly the same time. Further the dosage from can be designed based on the pharmacokinetic profiles where the peak plasma concentration of one compound, for example the quality of sleep improving agent, e.g., pilocarpine, cevimeline, anethole trithione, aclatonium napadisilate, and yohimbine, corresponds to maximum amount of poor sleep quality caused by the OAB drug, for example oxybutynin, tolterodine, solifenacin, darifenacin, trospium, fesoterodine, propiverine, imidafenacin, or dicyclomine.

Thus, some of the pharmaceutical compositions contemplated for use in the methods disclosed herein include, but are not limited to:

immediate release oxybutynin, tolterodine, solifenacin, darifenacin, trospium, fesoterodine, propiverine, imidafenacin, or dicyclomine, in combination with immediate release pilocarpine, cevimeline, anethole trithione, aclatonium napadisilate, or yohimbine;

delayed (whether sustained or extended) release oxybutynin, tolterodine, solifenacin, darifenacin, trospium, fesoterodine, propiverine, imidafenacin, or dicyclomine and delayed (whether sustained or extended) release pilocarpine, cevimeline, anethole trithione, aclatonium napadisilate, or yohimbine;

delayed (whether sustained or extended) release oxybutynin, tolterodine, solifenacin, darifenacin, trospium, fesoterodine, propiverine, imidafenacin, or dicyclomine and immediate release pilocarpine, cevimeline, anethole trithione, aclatonium napadisilate, or yohimbine;

immediate release oxybutynin, tolterodine, solifenacin, darifenacin, trospium, fesoterodine, propiverine, imidafenacin, or dicyclomine, and delayed (whether sustained or extended) formulation of pilocarpine, cevimeline, anethole trithione, aclatonium napadisilate, or yohimbine.

Besides reducing the unalleviated symptoms of poor quality of sleep experienced by those being treated for overactive bladder, the methods disclosed herein have additional advantages. Currently, the dose of treatment drugs, such as oxybutynin, is limited because of side effects. Some patients who suffer from overactive bladder cannot tolerate dosages that provide adequate therapy because of the adverse side effects. These patients continue to suffer from overactive bladder even though they take their medications, solely because the medication is not administered at an effective dose. By lowering the side effects using the methods and compositions disclosed herein, the patient can be prescribed to take treatment drugs, such as oxybutynin, at higher doses. These higher doses result in having a less active bladder and also result in an increase in intrinsic bladder capacity.

As discussed above, the methods disclosed herein improve the quality of sleep in a patient. Quality of sleep is a subjective criterion and cannot be objectively measured. However, there are methods in the art to effectively measure subjective criteria, such as pain, comfort, anxiety, sadness, and the like. A well-known method is termed “visual analog scale,” or VAS. In this method, subjects are shown a line scaled from 0 to 100 mm. Subjects are asked to rate the subjective criterion from 0-100 mm and make a mark on the line corresponding to their rating. For example, subjects are told that 100 mm on the line means a very good night sleep and 0 mm on the line means complete wakefulness. They should rate their quality of sleep on the line. Changes in the quality of sleep of a subject can then be measured using this technique throughout the treatment period.

In another aspect, the invention relates to a method of treating a patient comprising administering to a patient in need thereof a therapeutically effective amount of a pharmaceutical composition comprising a combination of an antimuscarinic or an anticholinergic agent, as described herein, and a muscarinic agonist, as described herein; and a physiologically acceptable carrier, diluent, or excipient, or a combination thereof.

The term “pharmaceutical composition” refers to a mixture of a compound of the invention with other chemical components, such as diluents, lubricants, bulking agents, disintegrant or carriers. The pharmaceutical composition facilitates administration of the compound to an organism. Multiple techniques of administering a compound exist in the art including, but not limited to, oral, injection, inhalation, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, transdermal, rectal, transmucosal, or intestinal administration; parenteral delivery, including intramuscular, subcutaneous, intravenous, intramedullary injections, as well as intravaginal, inhalation, intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained, extended, or delayed release formulation. In addition, one may administer the composition by transdermal approach or directly into the bladder.

The pharmaceutical compositions to be used in the method of treating a patient of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen and desired pharmacokinetic profiles of each component of combination therapy. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

The compounds may also be formulated in rectal compositions such as suppositories or retention enemas.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, and the like. Salts tend to be more soluble in aqueous or other protonic solvents than are the corresponding free acids or base forms.

Pharmaceutical compositions suitable for use in the method for treating a patient of the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated.

Typically, the daily dose range of the composition administered to the patient can be from about 0.5 to 1000 mg/kg of the patient's body weight. The daily dosage may be a single one or a series of two or more given in the course of one or more days, as is needed by the patient. Note that for almost all of the specific compounds mentioned in the present disclosure, human daily dosages for treatment of at least some condition have been established. For example, for oxybutynin, tolterodine, solifenacin, darifenacin, trospium, fesoterodine, propiverine, imidafenacin, and dicyclomine the preferred daily dosage is between 0.1 mg to 50 mg, and the more preferred daily dosage is between 0.2 mg to 30 mg. Other daily dose ranges include between 10 to 50 mg, between 20 to 50 mg, between 30 to 50 mg, between 40 to 50 mg, between 20 to 40 mg, between 10 to 20 mg, between 10 to 30 mg, between 20 to 30 mg, and between 30 to 40 mg. The daily dose may also be at 10 mg, 20 mg, 30 mg, 40 mg, or 50 mg. For pilocarpine, cevimeline, anethole trithione, aclatonium napadisilate, and yohimbine, the preferred daily dosage is between 0.1 mg to 100 mg, and the more preferred daily dosage is between 0.1 mg to 50 mg. Other daily dose ranges include between 10 to 50 mg, between 20 to 50 mg, between 30 to 50 mg, between 40 to 50 mg, between 20 to 40 mg, and between 30 to 40 mg. The daily dose may also be at 10 mg, 20 mg, 30 mg, 40 mg, or 50 mg.

Although the exact daily dosage can be determined on a drug-by-drug basis, in most cases, some generalizations regarding the dosage can be made. The daily dosage regimen for an adult human patient may be, for example, an oral dose of between 0.001 mg and 1000 mg of each ingredient, preferably between 0.01 mg and 500 mg, for example 1 to 200 mg or each ingredient of the pharmaceutical compositions of the present invention or a pharmaceutically acceptable salt thereof calculated as the free base or free acid, the composition being administered 1 to 3 times per day or per week. Alternatively the compositions of the invention may be administered by continuous such as sustained, delayed, or extended release, preferably at a dose of each ingredient up to 500 mg per day. Thus, the total daily dosage by oral administration of each ingredient will typically be in the range 0.1 mg to 2000 mg. Suitably the compounds will be administered for a period of continuous therapy, for example for a day, a week or more, or for months or years.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

It will be understood by those of skill in the art that numerous and various modifications can be made without departing from the spirit of the present invention. Therefore, it should be clearly understood that the forms of the present invention are illustrative only and are not intended to limit the scope of the present invention.

### EXAMPLES

The examples below are non-limiting and are merely representative of various aspects of the invention.

### Example 1

### Effect of Combination of Oxybutynin and Pilocarpine on Quality of Sleep in OAB Patients being Treated with Oxybutynin

A study was conducted to evaluate the effect of oxybutynin alone and in combination with pilocarpine versus placebo on sleep quality in patients who are already being treated for OAB by administration of oxybutynin for at least two months and who do not display overt OAB symptoms. The objective of the study is to determine quality of sleep after oral administration of oxybutynin, alone and in combination with pilocarpine, vs. placebo.

The study was a randomized, crossover, multi-center (seven centers), two-sequence and two-period study. Approximately 40 subjects whose OAB symptoms were controlled on immediate release oxybutynin (5 or 10 mg bid) were randomized to twice-daily oxybutynin monotherapy or twice-daily oxybutynin plus pilocarpine for 2 weeks in period 1. Subjects were then crossed over to the alternate treatment for 2 weeks in period 2. This crossover study was performed to have an intrasubject comparison of the effects of oxybutynin alone and in combination with pilocarpine on degree of difficulty of sleep.

When assigned to oxybutynin plus pilocarpine therapy, subjects were required to take their pilocarpine dose approximately 30 minutes after taking their oxybutynin dose. When assigned to oxybutynin alone, subjects were required to take their placebo approximately 30 minutes after taking their oxybutynin dose.

Randomization to period 1 treatment was made by a predetermined randomization schedule, prepared by a biostatistician and maintained at each clinical site. Once the subject was randomized, there was no adjustment in dosage, unless it was required in response to an adverse event or worsening OAB symptoms.

Enrollment of subjects already taking this medication was used to enable the collection of steady-state baseline information and to minimize discontinuations during the 4-week treatment period. On three consecutive days, subjects were asked to complete paper diaries where the subject answered questions related to the degree of difficulty of sleep and responded by using a VAS. These questions and VAS have been validated and used in other clinical studies. Furthermore, each subject relinquished the completed diary at the end of each treatment period following a review by clinic staff. In addition, study treatments were balanced and statistical analyses evaluated treatment sequence, baseline conditions to determine if the order in which the treatments were given influenced the results.

Subjects were given the study drug(s) and a diary to record their usage of medication. Subjects then followed the study or reference regimen for two weeks and were crossed over to the opposite treatment regimen for another two weeks.

The methods used to collect information for assessment of quality of sleep, subjects were asked specific questions and they rated their quality of sleep using a VAS. The mean of the values obtained on each of the three days was used as the sole value for the baseline or treatment value. The methods used to evaluate effect on quality of sleep of the combination and oxybutynin alone are widely used and considered standard.

The VAS is a validated scale utilized in clinical trials and registrational studies of many drugs. Patients were instructed on how to complete the diary over 3 consecutive days and perform VAS assessments for quality of sleep on the diary days. The ratings were from 0 to 100 on a 100-mm line, and the patient was asked to rate herself by marking the horizontal line. For instance, quality of sleep was quantified as 0=easy and 100=difficult.

All calculations and statistical analyses were performed using the SAS® statistical analysis system, version 9.1.3.

In Table 1, subject initials have been removed to further protect subject privacy. These table legend is:

(1) Treatment A: Oxybutynin (5 or 10 mg twice a day)
Treatment B: Oxybutynin+Pilocarpine (5 or 10 mg each twice a day)

(2) Subject discontinued during the treatment.

TABLE 1
Quality of Sleep

| | | | | | | Oxybutynin + | |
|---|---|---|---|---|---|---|---|
| | | Baseline | | Oxybutynin | | Pilocarpine | |
| Subj # | Seq (1) | Value | Count | Value | Count | Value | Count |
| 101 | BA(2) | 61.0 | 3 | ND | 0 | 29.3 | 3 |
| 102 | AB | 16.3 | 3 | 6.3 | 3 | 4.7 | 3 |
| 103 | AB | 15.3 | 3 | 15.7 | 3 | 29.7 | 3 |
| 104 | BA | 69.3 | 3 | 89.0 | 3 | 68.0 | 3 |
| 105 | AB | 47.7 | 3 | 45.3 | 3 | 66.3 | 3 |
| 106 | BA | 30.7 | 3 | 48.3 | 3 | 6.7 | 3 |
| 107 | BA | 0.0 | 1 | 0.0 | 1 | 6.0 | 1 |
| 200 | BA | 13.7 | 3 | 13.0 | 3 | 33.0 | 3 |
| 201 | AB | 65.0 | 3 | 76.7 | 3 | 52.0 | 3 |
| 202 | BA | 63.7 | 3 | 71.3 | 3 | 41.3 | 3 |
| 205 | AB | 38.7 | 3 | 28.3 | 3 | 36.0 | 3 |
| 206 | BA | 27.7 | 3 | 79.0 | 3 | 43.3 | 3 |
| 207 | BA | 63.3 | 3 | 72.0 | 3 | 46.0 | 3 |
| 210 | AB | 70.7 | 3 | 69.7 | 3 | 4.0 | 3 |
| 211 | AB | 54.0 | 3 | 61.7 | 3 | 58.7 | 3 |
| 212 | BA | 11.0 | 3 | 59.7 | 3 | 1.0 | 3 |
| 400 | BA | 32.3 | 3 | 17.7 | 3 | 34.3 | 3 |
| 405 | AB | 33.7 | 3 | 48.3 | 3 | 19.7 | 3 |
| 406 | AB | 51.3 | 3 | 61.0 | 3 | 54.0 | 3 |
| 407 | BA | 58.7 | 3 | 34.7 | 3 | 37.3 | 3 |
| 501 | AB | 11.3 | 3 | 40.3 | 3 | 11.7 | 3 |
| 502 | AB | 49.0 | 3 | 48.3 | 3 | 57.7 | 3 |
| 503 | BA | 67.0 | 3 | 92.7 | 3 | 70.3 | 3 |
| 504 | BA | 5.0 | 3 | 18.0 | 3 | 11.0 | 3 |
| 505 | AB | 24.7 | 3 | 9.7 | 3 | 1.3 | 3 |
| 506 | BA(2) | 27.7 | 3 | ND | 0 | 8.0 | 3 |
| 507 | BA | 25.0 | 3 | 31.3 | 3 | 62.7 | 3 |
| 508 | AB | 63.3 | 3 | 69.3 | 3 | 12.0 | 3 |
| 600 | AB | 13.7 | 3 | 25.7 | 3 | 18.3 | 3 |
| 602 | BA | 21.3 | 3 | 10.0 | 3 | 4.3 | 3 |
| 603 | BA(2) | 65.3 | 3 | ND | 0 | ND | 0 |
| 604 | AB | 28.7 | 3 | 8.3 | 3 | 18.0 | 3 |
| 605 | BA | 5.7 | 3 | 8.3 | 3 | 5.3 | 3 |
| 606 | AB | 25.7 | 3 | 30.3 | 3 | 5.0 | 3 |
| 607 | AB | 55.3 | 3 | 81.3 | 3 | 42.7 | 3 |
| 608 | AB | 78.3 | 3 | 63.7 | 3 | 31.3 | 3 |
| 610 | BA | 9.3 | 3 | 20.3 | 3 | 20.0 | 3 |
| 613 | BA | 37.3 | 3 | 61.3 | 3 | 25.3 | 3 |
| 702 | AB | 67.7 | 3 | 13.7 | 3 | 40.0 | 3 |
| 703 | BA | 53.7 | 3 | 36.7 | 3 | 29.0 | 3 |
| 709 | BA | 4.0 | 3 | 7.7 | 3 | 12.7 | 3 |
| 712 | AB | 26.3 | 3 | 54.7 | 3 | 56.3 | 3 |
| 713 | AB | 26.3 | 3 | 24.0 | 3 | 44.3 | 3 |
| mean | | 37.6 | 43 | 41.3 | 40 | 30.0 | 42 |
| stddev | | 22.7 | | 26.8 | | 21.1 | |

Count = number of diaries completed

The mean score for quality of sleep at baseline was 37.6 mm and remained unchanged at 41.3 mm during treatment with oxybutynin alone (Table 1). The combination of oxybutynin and pilocarpine decreased the degree of difficulty of sleep by an average of 7.6 mm to the final value of 30.0 mm, indicating that subjects felt improvement in ease of sleep compared to baseline. As expected, mean sleep quality after 2 additional weeks of oxybutynin monotherapy was unchanged from baseline (41.3 vs. 37.6). Combination therapy was associated with a surprising but modest decrease (to 30.0) relative to both baseline and oxybutynin alone. The VAS scores, on a 100-point mm scale, showed a statistically significantly better response (reduction from baseline) for the combination treatment than for the oxybutynin monotherapy for improving quality of sleep in patients being treated for OAB.

A summary of the least square mean (LSM) changes from baseline in quality of sleep is shown in Table 2.

TABLE 2
Change in LSM from Baseline in Quality
of Sleep in the ITT Population, N = 42

| Parameter (mm) | Combination | Oxybutynin | P value |
|---|---|---|---|
| Quality of Sleep* | −7.6 | 3.7 | 0.0073 |

*lower scores indicate improvement from baseline

Of the 43 subjects randomized 21 were randomized initially to treatment AB (A=oxybutynin alone, B=combination of oxybutynin and pilocarpine) and 22 to treatment BA. As noted in Table 3, the sequence of treatment did not make any difference in outcome. In this analysis, the LSM values for sequence AB and sequence BA are presented for each parameter. The p values represent the test of hypotheses that there were no differences between the two sequence means.

TABLE 3
Change in LSM from Baseline in Quality
of Sleep Sequence Effect, N = 42

| Parameter (mm) | Treatment AB | Treatment BA | P value |
|---|---|---|---|
| Quality of Sleep* | −1.86 | −1.66 | 0.6131 |

*lower scores indicate improvement from baseline

These findings clearly show that the order of treatments did not affect quality of sleep. Thus, these data support the view that despite the lack of blinding the outcome measures were not influenced by the order of treatments.

The results of this study were quite unexpected and surprising. There was no evidence that symptoms of OAB worsened when pilocarpine was added (approximately 30 minutes after oxybutynin) to a twice-daily regimen of IR oxybutynin. There was a small, but statistically significant decrease in urinary frequency when the antagonist and agonist were given together, compared to oxybutynin alone, supporting the view that pilocarpine does not adversely affect bladder function and the combination may have even improved OAB symptomatology (improved sleep quality). The number of urgency or urgency episodes did not change from baseline with the combination. Fluid intake was not different between the two treatments and thus provides additional evidence that the two agents are working preferentially at the bladder and salivary glands to provide the right balance of activity for improvement in OAB treatment.

### Example 2

### Effect of Combination of Oxybutynin and Cevimeline on Quality of Sleep in OAB Patients being Treated with Oxybutynin

A study is conducted to evaluate the effect of oxybutynin alone and in combination with cevimeline versus placebo on sleep quality in patients who are already being treated for OAB by administration of oxybutynin for at least two months and who do not display overt OAB symptoms. The objective of the study is to determine quality of sleep after oral administration of oxybutynin, alone and in combination with cevimeline, vs. placebo.

The study is a randomized, open-label, crossover, multi-center, two-sequence and two-period study. Approximately 40 subjects whose OAB symptoms are controlled on immediate release oxybutynin (5 or 10 mg bid) are randomized to twice-daily oxybutynin monotherapy (same dose) or twice-daily oxybutynin (same dose) plus cevimeline (30 mg daily) for 2 weeks in period 1. Subjects are then crossed over to the alternate treatment for 2 weeks in period 2. This crossover study is performed to have an intrasubject comparison of the effects of oxybutynin alone and in combination with cevimeline on degree of difficulty of sleep.

When assigned to oxybutynin plus cevimeline therapy, subjects are required to take their cevimeline dose either simultaneously with their oxybutynin dose, or 30 minutes after taking their oxybutynin dose. When assigned to oxybutynin alone, subjects were required to take their placebo either simultaneously with their oxybutynin dose, or 30 minutes after taking their oxybutynin dose.

Randomization to period 1 treatment is made by a predetermined randomization schedule, prepared by a biostatistician and maintained at each clinical site. Once the subject is randomized, there is no adjustment in dosage, unless it is required in response to an adverse event or worsening OAB symptoms.

Enrollment of subjects already taking this medication is used to enable the collection of steady-state baseline information and to minimize discontinuations during the 4-week treatment period. Subjects are asked to complete paper diaries where the subject answered questions related to the degree of difficulty of sleep and responded by using a visual analog scale (VAS). These questions and VAS have been validated and used in other clinical studies. Furthermore, each subject relinquishes the completed diary at the end of each treatment period following a review by clinic staff. In addition, study treatments are balanced and statistical analyses evaluated treatment sequence, baseline conditions to determine if the order in which the treatments are given influenced the results.

Subjects are given the study drug(s) and a diary to record their usage of medication. Subjects then follow the study or reference regimen for two weeks and are crossed over to the opposite treatment regimen for another two weeks.

The methods used to collect information for assessment of quality of sleep, subjects are asked specific questions and they rate their quality of sleep using VAS. The mean of the values obtained on each of the three days was used as the sole value for the baseline or treatment value. The method used to evaluate effect on quality of sleep of the combination and oxybutynin alone are widely used and considered standard.

The VAS is a validated scale utilized in clinical trials and registrational studies of many drugs. Patients are instructed on how to complete the diary over 3 consecutive days and perform VAS assessments for quality of sleep on the diary days. The ratings are from 0 to 100 on a 100-mm line, and the patient was asked to rate herself by marking the horizontal line. For instance, quality of sleep is quantified as 0=easy and 100=difficult.

All calculations and statistical analyses are performed using the SAS® statistical analysis system, version 9.1.3.

### Example 3

### Effect of Combination of Tolterodine and Pilocarpine on Quality of Sleep in OAB Patients being Treated with Tolterodine

The same procedure as that shown in Example 1 is followed except that the patients are treated with tolterodine instead of oxybutynin.

### Example 4

### Effect of Combination of Tolterodine and Cevimeline on Quality of Sleep in OAB Patients being Treated with Tolterodine

The same procedure as that shown in Example 2 is followed except that the patients are treated with tolterodine instead of oxybutynin.

### Example 5

### Combination of an Oxybutynin and a Pilocarpine for the Treatment of Poor Quality of Sleep in OAB Patient

A study is conducted to evaluate the effect of oxybutynin in combination with pilocarpine versus placebo on sleep quality in patients suffering from OAB. The same procedure as that of Example 1 is followed, except that the patients in this study are naïve to the treatment with antimuscarinic therapy, i.e., the patients have never been treated for their OAB by the administration of an antimuscarinic.

### Example 6

### Combination of an Oxybutynin and a Cevimeline for the Treatment of Poor Quality of Sleep in OAB Patient

The same procedure as that shown in Example 5 is followed except that the patients are treated with cevimeline instead of pilocarpine.

### Example 7

### Combination of a Tolterodine and a Pilocarpine for the Treatment of Poor Quality of Sleep in OAB Patient

The same procedure as that shown in Example 5 is followed except that the patients are treated with tolterodine instead of oxybutynin.

### Example 8

### Combination of a Tolterodine and a Cevimeline for the Treatment of Poor Quality of Sleep in OAB Patient

The same procedure as that shown in Example 6 is followed except that the patients are treated with tolterodine instead of oxybutynin.

### Example 9

### Effect of Combination of Imidafenacin and Pilocarpine on Quality of Sleep in OAB Patients being Treated with Tolterodine

The same procedure as that shown in Example 1 is followed except that the patients are treated with imidafenacin (0.1 mg) instead of oxybutynin.

### Example 10

### Effect of Combination of Imidafenacin and Cevimeline on Quality of Sleep in OAB Patients being Treated with Tolterodine

The same procedure as that shown in Example 2 is followed except that the patients are treated with imidafenacin (0.1 mg) instead of oxybutynin.

### Example 11

### Combination of an Imidafenacin and a Pilocarpine for the Treatment of Poor Quality of Sleep in OAB Patient

A study is conducted to evaluate the effect of imidafenacin (0.1 mg) in combination with pilocarpine versus placebo on sleep quality in patients suffering from OAB. The same procedure as that of Example 1 is followed, except that the patients in this study are naïve to the treatment with antimuscarinic therapy, i.e., the patients have never been treated for their OAB by the administration of an antimuscarinic.

### Example 12

### Combination of an Imidafenacin and a Cevimeline for the Treatment of Poor Quality of Sleep in OAB Patient

The same procedure as that shown in Example 9 is followed except that the patients are treated with cevimeline instead of pilocarpine.

### Example 13

### Combinations for the Treatment of Poor Quality of Sleep in OAB Patient

The same procedure as that shown in Example 1 is followed except that the patients are treated with one of the following drug combinations:

oxybutynin and anethole trithione, oxybutynin and aclatonium napadisilate, oxybutynin and yohimbine, tolterodine and anethole trithione, tolterodine and aclatonium napadisilate, tolterodine and yohimbine, solifenacin and pilocarpine, solifenacin and cevimeline, solifenacin and anethole trithione, solifenacin and aclatonium napadisilate, solifenacin and yohimbine, darifenacin and pilocarpine, darifenacin and cevimeline, darifenacin and anethole trithione, darifenacin and aclatonium napadisilate, darifenacin and yohimbine, trospium and pilocarpine, trospium and cevimeline, trospium and anethole trithione, trospium and aclatonium napadisilate, trospium and yohimbine, fesoterodine and pilocarpine, fesoterodine and cevimeline, fesoterodine and anethole trithione, fesoterodine and aclatonium napadisilate, fesoterodine and yohimbine, propiverine and pilocarpine, propiverine and cevimeline, propiverine and anethole trithione, propiverine and aclatonium napadisilate, propiverine and yohimbine, imidafenacin and anethole trithione, imidafenacin and aclatonium napadisilate, imidafenacin and yohimbine, dicyclomine and pilocarpine, dicyclomine and cevimeline, dicyclomine and anethole trithione, dicyclomine and aclatonium napadisilate, and dicyclomine and yohimbine.

What is claimed is:

1. A method of improving quality of sleep in a patient suffering from overactive bladder, the method comprising:
(a) identifying a patient in need thereof, and
(b) administering to the patient a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof,
wherein the first compound is an antimuscarinic or an anticholinergic agent and the second compound is a muscarinic agonist,
and whereby the quality of sleep in the patient is improved.

2. The method of claim 1, wherein the first compound is selected from the group consisting of oxybutynin, tolterodine, solifenacin, darifenacin, trospium, fesoterodine, propiverine, imidafenacin, and dicyclomine.

3. The method of claim 1, wherein the second compound is selected from the group consisting of pilocarpine, cevimeline, anethole trithione, aclatonium napadisilate, and yohimbine.

4. The method of claim 1, wherein the first compound and the second compound are administered nearly simultaneously.

5. The method of claim 1, wherein the first compound is administered prior to the second compound.

6. The method of claim 1, wherein the first compound and the second compound are together disposed in the same dosage form.

7. The method of claim 1, wherein the first compound is administered at a daily dose of between 0.1 mg to 50 mg.

8. The method of claim 1, wherein the second compound is administered at a daily dose of between 0.1 mg to 100 mg.

9. A method of improving quality of sleep in a patient suffering from nocturnia, the method comprising:
(a) identifying a patient in need thereof; and
(b) administering to the patient a therapeutically effective amount of a first compound, a free base thereof or a pharmaceutically acceptable salt or prodrug thereof, and a therapeutically effective amount of a second compound, a free base thereof or a pharmaceutically acceptable salt or prodrug thereof,
wherein the first compound is an antimuscarinic or an anticholinergic agent, the second compound is a muscarinic agonist, and
whereby the quality of sleep in the patient is improved.

10. The method of claim 9, wherein the first compound is selected from the group consisting of oxybutynin, tolterodine, solifenacin, darifenacin, trospium, fesoterodine, propiverine, imidafenacin, and dicyclomine.

11. The method of claim 9, wherein the second compound is selected from the group consisting of pilocarpine, cevimeline, anethole trithione, aclatonium napadisilate, and yohimbine.

12. The method of claim 9, wherein the first compound is administered at a daily dose of between 0.1 mg to 50 mg.

13. The method of claim 9, wherein the second compound is administered at a daily dose of between 0.1 mg to 100 mg.

14. A method of improving quality of sleep in a patient being treated for overactive bladder by administration of a first compound, the method comprising:
(a) identifying a patient in need thereof, and
(b) administering to the patient a therapeutically effective amount of a second compound, while continuing the administration of therapeutically effective amount of the first compound,
wherein the first compound is an antimuscarinic agent or an anticholinergic agent,
wherein the second compound is a muscarinic agonist, and
whereby the quality of sleep in the patient is improved.

15. The method of claim 14, wherein the patient suffers from nocturnia.

16. The method of claim 14, wherein the first compound is selected from the group consisting of oxybutynin, tolterodine, solifenacin, darifenacin, trospium, fesoterodine, propiverine, imidafenacin, and dicyclomine.

17. The method of claim 14, wherein the second compound is selected from the group consisting of pilocarpine, cevimeline, anethole trithione, aclatonium napadisilate, and yohimbine.

18. The method of claim 14, wherein the first compound is administered at a daily dose of between 0.1 mg to 50 mg.

19. The method of claim 14, wherein the second compound is administered at a daily dose of between 0.1 mg to 100 mg.
